(19) United States
(12) Patent Application Publication
(10) Pub. No.: US 2025/0177688 A1
(43) Pub. Date: Jun. 5, 2025

(54) WIRE SPOOL ROTATION SYSTEM

(71) Applicant: BIOSENSE WEBSTER (ISRAEL) LTD., Yokneam (IL)

(72) Inventors: Assaf Govari, Haifa (IL); Ilya Sitnitsky, Nahariya (IL); Alexander Shechtman, Haifa (IL)

(21) Appl. No.: 18/947,018

(22) Filed: Nov. 14, 2024

Related U.S. Application Data
(60) Provisional application No. 63/606,110, filed on Dec. 5, 2023.

(51) Int. Cl. A61M 25/00; A61B 5/287; A61B 18/14; B25J 9/14; B25J 13/08

(52) U.S. Cl. CPC A61M 25/0009; B25J 9/14; B25J 13/085; A61B 5/287; A61B 18/1492; A61M 2207/10

(57) ABSTRACT

A system for manufacturing a medical device, the system includes a wire spool, a pulling device, a wire-tension meter, an air-blowing device, and a controller. The wire spool is configured to supply wire, the wire spool being configured to be rotated by (i) pulling of the wire and (ii) an airflow applied onto the wire spool. The pulling device is configured to pull the wire from the spool. The wire-tension meter is configured to measure a tension of the pulled wire. The air-blowing device is configured to apply the airflow at a controllable airflow rate. The controller is configured to receive tension readings from the wire-tension meter and to adjust the airflow rate provided by the air-blowing device based on tension readings.

### CROSS-REFERENCE TO RELATED APPLICATIONS

This application is related to U.S. Provisional Patent Application 63/606,110, filed Dec. 5, 2024, whose disclosure is incorporated herein by reference.

### TECHNICAL FIELD

The present disclosure relates generally to the manufacturing of medical devices, and particularly to systems and methods for wiring medical devices using wire spool rotation.

### BACKGROUND OF THE DISCLOSURE

The manufacturing of invasive medical probes may be a delicate and elaborate process. In particular, the limitation on size makes the electrical wires extending through a shaft of some probes very delicate to assemble. The outcome is time-consuming manual labor in manufacturing some probes, like the multi-wired cardiac mapping electroanatomical (EA) mapping catheters. It is desired to automate the assembly process of such probes. However, automation should accommodate the delicate nature of the wires.

The present disclosure will be more fully understood from the following detailed description of the examples thereof, taken together with the drawings in which:

### BRIEF DESCRIPTION OF THE DRAWINGS

FIG. 1 is a schematic, pictorial illustration of a catheter-based electroanatomical (EA) mapping and ablation system comprising wiring for multiple purposes, in accordance with an example of the present disclosure;

FIG. 2 is a diagram that schematically illustrates a manufacturing system configured to supply wire for wiring a shaft of a medical device, in accordance with an example of the present disclosure;

FIG. 3 is a diagram that schematically illustrates a manufacturing system configured to supply multiple wires for wiring a shaft of a medical device, in accordance with an example of the present disclosure; and

FIG. 4 is a flow chart that schematically illustrates a manufacturing method for supplying wire for wiring a shaft of the medical device, in accordance with an example of the present disclosure.

### DETAILED DESCRIPTION OF EXAMPLES

### Overview

Some medical devices, such as invasive medical probes, may incorporate long, delicate wiring. For example, cardiac diagnostic probes, e.g., cardiac catheters, can include distal assemblies comprising multiple electrodes (e.g., 100), where each electrode is connected to a dedicated thin (e.g., 10 microns) electrical wire. Each wire is extended through a shaft of the probe and connected at the proximal end to electrical circuitry.

Currently, electrical wiring is done by hand with a preassembled cable. It is desired to automate the wiring process by proposing a system that will pull wires from a spool and position the wires over its dedicated connection for establishing electrical connections between pad electrodes and sensors at the distal assembly of the catheter and a connector at the proximal end of the catheter.

During a possibly attempted at least semi-automated wiring of the probes, wires will be pulled from wire spools using a dedicated wiring-pulling system (e.g., with a motorized gripper). The grip on the wire from the spool needs to be maintained at a defined tension. The wire is very delicate, and the inertia of the spool adds tension to the wire as it is being pulled, which can cause the wire to tear. As the wires used in probes are typically thin and delicate, their assembly requires a very careful manufacturing process, which despite possible efforts to at least semi-automate can be very lengthy and costly.

Examples of the present disclosure that are described hereinafter provide automated techniques to maintain, up to predefined tolerances, a predefined pulled wire speed while maintaining a predefined wire tension. To this end, the technique controls spool rotation with an air-blowing device, which can be a blower, a fan, or any other suitable device capable of blowing air. In one example, blades are fixed to a spool that can freely rotate on bearings coupled to a spool shaft. Airflow is then applied to the blades to assist (e.g., adjust) the rotation of the spool as the wire is gripped and pulled from the spool. In another example, a disk-shaped brush may be used in place of the blades and the airflow is applied on the brush to rotate the spool.

The tension on the wire is controlled using a feedback loop that receives input from one or more tension meters. Based on the input from the meters, a processor can control the airflow rate of the air blower. The maximum tension tolerated is used as an input to set the speed of the manufacturing process. The processor can therefore operate the system at the maximum possible speed, up to a predefined tolerance, while maintaining the tension below a predefined maximal tension level.

Additionally, or alternatively, the controller is configured to receive input from the pulling device and to adjust the airflow rate provided by the air-blowing device based on both a tension reading and input from the pulling device. The information received from the pulling device includes pulling start time, stop time, and, optionally, pulling rate. Based on the information, the controller can also alter the pulling rate based on the detected tension.

The disclosed technique can reduce most of the lengthy and costly stages of the assembly of some medical devices, such as invasive probes.

### System Description

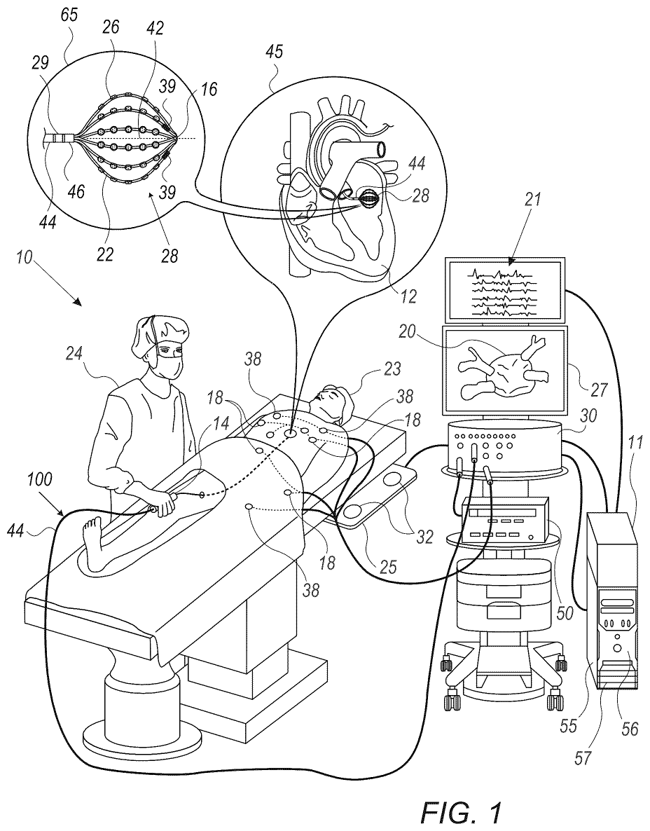

FIG. 1 is a schematic, pictorial illustration of a catheter-based electroanatomical (EA) mapping and ablation system 10 comprising multiple-purpose wiring 100, in accordance with an example of the present disclosure. Techniques for manufacturing wiring 100 are described further below.

System 10 includes one or more catheters, which are percutaneously inserted by physician 24 through the patient's vascular system into a chamber or vascular structure of a heart 12. The one or more catheters may include catheters dedicated for sensing intracardiac electrogram (IEGM) signals, catheters dedicated for ablating and/or catheters dedicated for both sensing and ablating. An example basket catheter 14, configured for sensing IEGM, is illustrated herein. As seen in inset 45, physician 24 brings a basket type of expandable distal end assembly 28 (also called hereinafter “expandable distal-end assembly 28”) fitted on a shaft 44 of catheter 14 into contact with the heart wall for sensing a target site in heart 12. For ablation, physician 24 similarly brings a distal end of an ablation catheter to a target site for ablating. Assembly 28 has a longitudinal axis 42 and can be deflected relative to a longitudinal axis defined of distal end 46 of shaft 44.

As seen in inset 65, catheter 14 is an exemplary catheter that includes multiple electrodes 26 optionally distributed over a plurality of splines 22 at expandable distal-end assembly 28 and configured to sense IEGM signals. Catheter 14 additionally includes (i) a proximal position sensor 29 embedded in distal end 46 of shaft 44 near expandable distal end assembly 28, and (ii) two distal position sensors 39 to track the position of the distal end of expandable distal end assembly 28. Optionally, and preferably, position sensors 29 and 39 are magnetic-based position sensors that include magnetic coils for sensing three-dimensional (3D) positions. Distal end 46 of shaft 44 may comprise an amplifying circuit configured to amplify the output from sensors 29 and 39.

System 10 includes one or more electrode patches 38 positioned for skin contact on patient 23 to establish a location reference for location pad 25 as well as impedance-based tracking of electrodes 26. For impedance-based tracking, electrical current is directed toward electrodes 26 and sensed at electrode skin patches 38, such that the location of each electrode can be triangulated via electrode patches 38.

The signals from the electrodes and various sensors are conveyed via multiple-purpose wires 100 extended through shaft 44. Most of these wires are thin and delicate, requiring a careful manufacturing process of catheter 14.

System 10 may include a recorder 11 that displays electrograms 21 captured with body surface ECG electrodes 18 and intracardiac electrograms (IEGM) captured with electrodes 26 of catheter 14. Recorder 11 may include pacing capability for pacing the heart rhythm and/or may be electrically connected to a standalone pacer.

System 10 may include an ablation energy generator 50 that is adapted to conduct ablative energy to one or more electrodes at a distal tip of a catheter configured for ablating. Energy produced by ablation energy generator 50 may include, but is not limited to, radiofrequency (RF) energy or pulsed-field ablation (PFA) energy, including monopolar or bipolar high-voltage DC pulses that may be used to effect irreversible electroporation (IRE), or combinations thereof.

Patient interface unit (PIU) 30 is configured to establish electrical communication between catheters, electrophysiological equipment, power supply and a workstation 55 for controlling the operation of system 10. Electrophysiological equipment of system 10 may include, for example, multiple catheters, location pad 25, body surface ECG electrodes 18, electrode patches 38, ablation energy generator 50, and recorder 11. Optionally, and preferably, PIU 30 additionally includes processing capability for implementing real-time computations of catheter locations and for performing ECG calculations.

Workstation 55 includes memory 57, processor unit 56 with memory or storage with appropriate operating software loaded therein, and user interface capability. Workstation 55 may provide multiple functions, optionally including (i) modeling endocardial anatomy in three-dimensions (3D) and rendering the model or anatomical map 20 for display on a display device 27, (ii) displaying activation sequences (or other data) compiled from recorded electrograms 21 on display device 27 in representative visual indicia or imagery superimposed on the rendered anatomical map 20, (iii) displaying real-time location and orientation of multiple catheters within the heart chamber, and (iv) displaying sites of interest, such as places where ablation energy has been applied, on display device 27. One commercial product embodying elements of system 10 is available as the CARTO™ 3 System, available from Biosense Webster, Inc., 31A Technology Drive, Irvine, CA 92618.

### Air Flow Assisted Wire Spool Rotation Manufacturing System

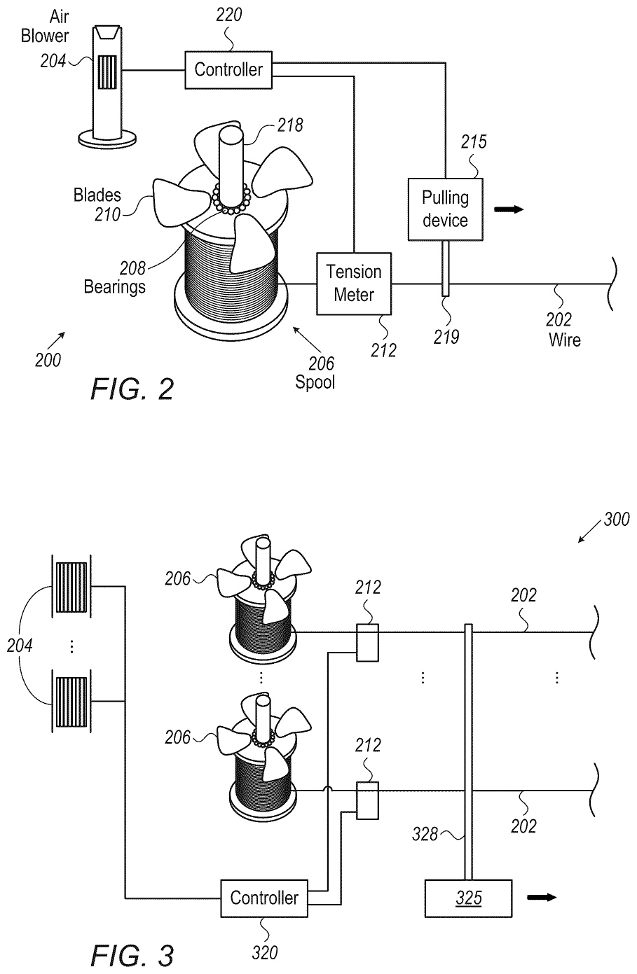

FIG. 2 is a diagram that schematically illustrates a manufacturing system 200 configured to supply wire 202 for wiring a shaft of a medical device, in accordance with an example of the present disclosure. System 200 can be used, for example, for manufacturing wiring 100 in shaft 44 of catheter 14 of FIG. 1.

System 200 comprises a wire spool 206 that can freely rotate on bearings 208 over a spool-shaft 218 on which the wire spool 206 is mounted. In the shown example, wire spool 206 is fitted with blades 210 that, with the airflow from an air blower 204, assist in the rotation of spool 206.

In some examples, wire 202 is pulled using a pulling device 215 that includes a gripper 219 and a stage. The motorized gripper is moved back and forth linearly in an automated controlled manner by the stage of pulling device 215. At certain locations over the wire, a welding or soldering tool (not shown) welds/solders the wire to, for example, a PCB board of the medical device.

The wire 202 is fed through a tension meter 212 that measures the tension of the wire and outputs readings to a controller 220. Additionally, or alternatively, the controller is configured to receive input from the pulling device. The information received from the pulling device includes pulling start time, stop time, and, optionally, the pulling rate.

As noted above, the pulling device 215 is configured to pull the wire off the wire spool as part of the manufacturing process of a medical device, such as an invasive probe. Wire 202, however, is too thin to withstand the tension applied by the pulling device 215 without the assistance provided by the closed-loop feedback mechanism of system 200. The assistance comes in the form of air blower 204 which is configured to apply a controllable rotation force to the wire spool by generating airflow at a controllable rate. Controller 220 is configured to control air blower 204 based on tension readings from wire-tension meter 212 and/or information from the pulling device 215, so that the wire spool is rotated at a predefined speed while maintaining a predefined tension on the wire. To this end, controller 220 adjusts the airflow rate provided by the air-blowing device (e.g., based on tension readings and the input from the pulling device).

FIG. 3 is a diagram that schematically illustrates a manufacturing system 300 configured to supply multiple wires 202 for wiring a shaft of a medical device, in accordance with an example of the present disclosure. The wires 202 are fed (each pulled from a respective wire spool 206) through respective tension meters 212 that measure the tension of each wire and output readings to a controller 320.

The controller 320 is configured to control tension on multiple wires 202 (e.g., 10 wires at a time). In some examples, wires 202 are pulled all at once using a pulling device 325 that includes a gripper 328 and a stage. The motorized gripper is moved back and forth linearly in an automated controlled manner by the stage of pulling device 325.

Controller 320 is configured to control individually each air blower 204 based on tension readings from respective wire-tension meters 212.

### Manufacturing Method for Supplying Wire Using Wire Air Flow Assisted Spool Rotation

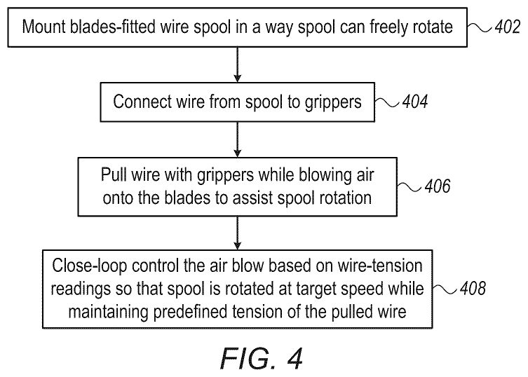

FIG. 4 is a flow chart that schematically illustrates a manufacturing method for supplying wire (e.g., wire 202) for wiring a shaft (e.g., shaft 44) of the medical device (e.g., catheter 14), in accordance with an example of the present disclosure. The process begins with mounting a selected spool fitted with blades to be rotated by an air blower of system 200, in a spool selection step 402. The spool is mounted on a spool shaft in a way that the spool can freely rotate, as described above.

Next, in a wire connecting step 404, a user (or a machine) connects the wire from the spool to the gripper of the system (e.g., of the pulling device) so that it can pull the wire as required during the medical device assembly session.

In a pulling wire step 406, manufacturing system 200 pulls the wire off of the spool with the assistance of the air blower that rotates the heavy spool, thus preventing tearing in the wire.

In a closed-loop spool rotation control step 408, controller 220 of system 200 controls air blower 208 based on wire-tension readings from tension meter 212 so that the spool is rotated to provide wire at a target speed while maintaining predefined tension in the pulled wire.

The flow chart is brought by way of example, where other steps or components may be added or replace existing ones.

### EXAMPLES

### Example 1

A system (200) for manufacturing a medical device, the system includes a wire spool (206), a pulling device (215), a wire-tension meter (212), an air-blowing device (204), and a controller (220). The wire spool (206) is configured to supply wire (202), the wire spool being configured to be rotated by (i) pulling of the wire (202) and (ii) an airflow applied onto the wire spool. The pulling device (215) is configured to pull the wire (202) from the spool (206). The wire-tension meter (212) is configured to measure a tension of the pulled wire (202). The air-blowing device (204) is configured to apply the airflow at a controllable airflow rate. The controller (220) is configured to receive tension readings from the wire-tension meter (212) and to adjust the airflow rate provided by the air-blowing device (204) based on tension readings.

### Example 2

The system (200) according to example 1, wherein the wire spool (206) is coupled to a bearing (208) configured to move on a spool-shaft (218) on which the wire spool (206) is mounted.

### Example 3

The system (200) according to any of examples 1 and 2, and comprising blades (210) fixed to the wire spool (206), the blades (210) configured to be rotated by the airflow.

### Example 4

The system (200) according to any of examples 1 and 2, and comprising a disk shaped brush fixed to the wire spool (206), the blades configured to be rotated by the airflow.

### Example 5

The system according to any of examples 1 through 4, wherein the controller is configured to maintain, up to a predefined tolerance, a predefined wire speed while maintaining a predefined wire tension.

### Example 6

The system according to any of examples 1 through 5, wherein the controller is further configured to receive input from the pulling device and to adjust the airflow rate provided by the air-blowing device based on the input from the pulling device.

### Example 7

The system according to any of examples 1 through 6, wherein the pulling device (215) comprises a gripper (219).

### Example 8

The system according to any of examples 1 through 7, and comprising a plurality of wire spools (206), wherein the pulling device (325) is configured to simultaneously pull a plurality of wires (202), and further comprising respective air-blowing devices (204), wherein the controller (320) is configured to individually control each of the air-blowing devices (204) to maintain, based on tension readings from the respective wire-tension meters (212), a constant tension on each of the wires (202) as it is being pulled.

### Example 9

The system according to any of examples 1 through 8, wherein the medical device is an invasive probe (14).

### Example 10

The system according to any of examples 1 through 9, wherein the wire (202) is an electrical wire.

### Example 11

A method for manufacturing a medical device, the method includes supplying wire (202) using a wire spool (206) configured to be rotated by (i) pulling of the wire and (ii) an airflow applied onto the wire spool (206). The wire (202) is pulled from the spool (206) using a pulling device (215). A tension of the pulled wire is measured using a wire-tension meter (212). The airflow is applied at a controllable airflow rate using an air-blowing device (204). Using a controller (220), tension readings are received from the wire-tension meter (212) and the airflow rate provided by the air-blowing device is adjusted based on tension readings.

Although the examples disclosed herein mainly address phacoemulsification procedures, the methods and systems disclosed herein can also be used in other applications, such as in vitrectomy surgery and other ophthalmic surgical procedures.

It will thus be appreciated that the examples described above are not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system for manufacturing a medical device, the system comprising:
a wire spool configured to supply wire, the wire spool configured to be rotated by (i) pulling of the wire and (ii) an airflow applied onto the wire spool;
a pulling device, configured to pull the wire from the spool;
a wire-tension meter configured to measure a tension of the pulled wire;
an air-blowing device, configured to apply the airflow at a controllable airflow rate; and
a controller, configured to receive tension readings from the wire-tension meter and to adjust the airflow rate provided by the air-blowing device based on tension readings.

2. The system according to claim 1, wherein the wire spool is coupled to a bearing configured to move on a spool-shaft on which the wire spool is mounted.

3. The system according to claim 1, and comprising blades fixed to the wire spool, the blades configured to be rotated by the airflow.

4. The system according to claim 1, and comprising a disk shaped brush fixed to the wire spool, the blades configured to be rotated by the airflow.

5. The system according to claim 1, wherein the controller is configured to maintain, up to a predefined tolerance, a predefined wire speed while maintaining a predefined wire tension.

6. The system according to claim 1, wherein the controller is further configured to receive input from the pulling device and to adjust the airflow rate provided by the air-blowing device based on the input from the pulling device.

7. The system according to claim 1, wherein the pulling device comprises a gripper.

8. The system according to claim 1, and comprising a plurality of wire spools, wherein the pulling device is configured to simultaneously pull a respective plurality of wires from the wire spools, and further comprising respective air-blowing devices, wherein the controller is configured to individually control each of the air-blowing devices based on tension readings from the respective wire-tension meters, to maintain a constant tension on each of the wires as it is being pulled.

9. The system according to claim 1, wherein the medical device is an invasive probe.

10. The system according to claim 1, wherein the wire is an electrical wire.

11. A method for manufacturing a medical device, the method comprising:
supplying wire using a wire spool configured to be rotated by (i) pulling of the wire and (ii) an airflow applied onto the wire spool;
pulling the wire from the spool using a pulling device;
measuring a tension of the pulled wire using a wire-tension meter;
applying the airflow at a controllable airflow rate using an air-blowing device; and
using a controller, receiving tension readings from the wire-tension meter and adjusting the airflow rate provided by the air-blowing device based on tension readings.

12. The method according to claim 11, wherein pulling the wire from the spool comprises using a wire spool that is coupled to a bearing configured to move on a spool-shaft on which the wire spool is mounted.

13. The method according to claim 11, wherein applying the airflow comprises applying the airflow to blades fixed to the wire spool, the blades configured to be rotated by the airflow.

14. The method according to claim 11, wherein applying the airflow comprises applying the airflow to a disk shaped brush fixed to the wire spool, the blades configured to be rotated by the airflow.

15. The method according to claim 11, wherein the controller is configured to maintain, up to a predefined tolerance, a predefined wire speed while maintaining a predefined wire tension.

16. The method according to claim 11, wherein using the controller further comprises receiving input from the pulling device and adjusting the airflow rate provided by the air-blowing device based on the input from the pulling device.

17. The method according to claim 11, wherein pulling the wire from the spool using a pulling device comprises a using a gripper.

18. The method according to claim 11, and comprising, using the pulling device, simultaneously pulling a plurality of wires from a respective plurality of wire spools using respective air-blowing devices, and further comprising individually controlling each of the air-blowing devices by the controller based on tension readings from the respective wire-tension meters, to maintain a constant tension on each of the wires as it is being pulled.

19. The method according to claim 11, wherein the medical device is an invasive probe.

20. The method according to claim 11, wherein the wire is an electrical wire.
